(19) United States
(12) Patent Application Publication
(10) Pub. No.: US 2025/0118011 A1
(43) Pub. Date: Apr. 10, 2025

(54) SYSTEM FOR VISUALIZING OCT SIGNALS

(71) Applicant: CARL ZEISS MEDITEC AG, Jena (DE)

(72) Inventors: Nassir NAVAB, Munich (DE); Michael SOMMERSPERGER, Munich (DE); Shervin DEHGHANI, Munich (DE)

(21) Appl. No.: 18/906,422

(22) Filed: Oct. 4, 2024

(30) Foreign Application Priority Data
Oct. 6, 2023 (DE) 102023127251.9

(51) Int. Cl. G06T 15/08; A61B 90/20; G06T 7/10; G06T 7/70; G06T 15/06

(52) U.S. Cl. CPC G06T 15/08; A61B 90/20; G06T 7/10; G06T 7/70; G06T 15/06; G06T 2200/24; G06T 2207/10016; G06T 2207/10024; G06T 2207/10056; G06T 2207/10101; G06T 2207/20092; G06T 2207/30004; G06T 2210/21; G06T 2210/41

(57) ABSTRACT

The present invention relates to a system for visualizing OCT signals, having a display means designed for the time-resolved display of image data and a control unit, the control unit being configured to receive a time-resolved OCT signal of a selected field of view of a sample from an OCT system, to ascertain, on the basis of the OCT signal, a time-resolved OCT image having at least one object and having a virtual surface, with a reflection of the at least one object in the OCT image being ascertained on the virtual surface, and to control the display means to display the time-resolved OCT image on the display means. The present invention also relates to a corresponding method for visualizing OCT signals.

### SUBJECT MATTER OF THE INVENTION

The present invention relates to a system for visualizing OCT signals, in particular for visualizing time-resolved OCT signals in the form of time-resolved OCT images, containing at least one object and a virtual surface. The present invention also relates to a method for correspondingly visualizing OCT signals.

### TECHNOLOGICAL BACKGROUND

The use of technological aids is part and parcel of modern medicine. By now, imaging methods and robotic systems are used equally as a matter of course in both surgery and diagnostics. In this context, the use of imaging methods allows the presentation and discrimination of various structures in the patient, and the image data obtained from the patient can be used advantageously in diagnostics and also in therapeutic and surgical methods.

For example, image data from a patient allows not only a surgeon to plan a surgical procedure better, but also assists them in performing the procedure. Robotic visualization systems are used to assist surgeons when performing surgical procedures. Said systems generally comprise at least one camera for recording images of the region to be operated on, with said camera being carried by a stand with an articulated structure. The stand allows the camera to be positioned relative to the subject by way of translational and/or rotational movements in order to capture images of a desired field of view (FOV) of the region to be operated on. In this context, the use of optical stereo cameras allows the capture of 3D image data.

In addition to the capture of surface information from a desired field of view, for example on the basis of reflected or back-scattered visible light, methods for capturing depth information from the field of view have also been developed in the meantime. These methods comprise optical coherence tomography (OCT), which allows the three-dimensional microscopic imaging of optically transparent and/or reflective objects and hence the recording of volume images of the biological tissue in the observed field of view. Optical coherence tomography (OCT) essentially is an interferometric method using broadband light with a short coherence length. As a rule, systems for capturing OCT data therefore comprise an interferometer and a broadband light source with a spectral width of more than 1% of the central wavelength.

OCT data can be captured sequentially or in parallel. For example, sequential capture of OCT data is implemented by virtue of a low-coherent source light beam being split at a beam splitter into a sample beam and a reference beam which are sent through two arms of an interferometer, with a movable reference mirror being arranged in the reference beam path and the object to be examined being arranged in the object beam path. A path difference between object beam and reference beam, and hence the measured depth, can be set by displacing the reference mirror. The object beam is raster-scanned over the sample in two dimensions by means of a mirror in the object beam path, allowing three-dimensional scanning of the sample as a result.

In the context of such a capture of OCT data in the time domain (time domain OCT-TD OCT), the spectral width of the light source Δλ corresponds to a coherence length L_{c} of L_{c}=λ*/ΔX. The axial resolution of an OCT system corresponds to the coherence length L_{c} of the utilized light and denotes the capability of resolving objects which have a spacing of at least the coherence length along the optical axis. For example, a light source in the near infrared range with a central wavelength of 800 nm and a spectral width of 80 nm has a coherence length of 7 μm, and an OCT system having such a source consequently has an axial resolution of approximately 1-10 μm. The transverse resolution of an OCT system is determined by the optical unit used in the object beam path, in particular by the object lens focusing the light on the object to be examined.

A sequential capture of OCT data is also possible in the frequency domain (frequency domain OCT-FD OCT), with a distinction generally being made between the use of a tunable source (swept source OCT) and the use of a dispersive detector (spectral domain OCT-SD OCT). In swept source OCT, the frequency of the excitation light source, for example a laser, is tuned, whereby it is possible to vary a path difference between sample beam and reference beam, and hence the scanned sample depth, even without a displaceable reference mirror. A broadband light source is likewise used in the case of SD OCT, but the detection is preceded by a separation of the frequency components of the interference signal, for example by an optical grating.

Slice and volume data of biological tissue can be captured by means of OCT, and this can significantly increase the information content for a surgeon. Consequently, an integration of OCT in surgical microscopes is desirable in order to be able to display both video data of the surface of a desired field of view and depth and/or slice images of the field of view, for example in simultaneous and/or overlaid fashion. Since surgeons are predominantly used to working with (sometimes three-dimensional) video image data to date, the use of overlaid two-dimensional OCT data or OCT images means a change for them. This could overwork the surgeons in the context of OCT image interpretation, and this could possibly even lead to incorrect interpretations, for example if surgeons were to interpret the OCT images just like video image data.

Moreover, OCT images are ascertained from OCT signals by calculation, for example by means of volume rendering, ray tracing, and/or ray marching. These methods inherently offer more varied possibilities for the image creation than the methods or algorithms usually used to create the (sometimes three-dimensional) video image data from the image signals captured by a surgical microscope. Thus, the impetus is to develop methods for creating OCT images (or for visualizing OCT signals) which, from the various possibilities, select those that optimally meet the demands from medical practice.

In particular, intraoperative optical coherence tomography, preferably using swept source OCT (ss-iOCT), i.e. volumetric imaging in real time, allows intraoperative 3D imaging virtually at video frame rates such as during surgical procedures in ophthalmology. The primary method for displaying volumetric OCT is direct volume rendering (DVR), since it enables the visualization of surgical procedures in a four-dimensional manner (4D-OCT) from desired viewing angles. In this way, an optimum viewing angle can be chosen for each surgical task. Conventionally, the operating site is viewed from a top-down perspective, which is restricted by the visual access of the surgical microscope. Side views in 4D-OCT, by contrast, make hand-eye coordination more difficult and increase cognitive burden for a surgeon due to viewing the procedure from an unusual angle.

The object of the present invention is therefore that of providing an improved system and an improved method for visualizing OCT signals, which overcomes or at least reduces the disadvantages of the prior art and makes the correct interpretation of OCT images easier to a surgeon or user.

### DESCRIPTION OF THE INVENTION

The object according to the invention is achieved by the subjects of the independent patent claims. Preferred developments are the subject matter of the dependent claims.

A first aspect of the present disclosure relates to a system for visualizing OCT signals, in particular for visualizing such signals captured by means of a medical device, for example a surgical microscope. In this case, the system according to the invention comprises a display means designed for the time-resolved display of image data. The display means preferably is one or more electronic visual displays, for example at least one electronic visual display of a surgical microscope, an electronic visual display fixedly installed within an operating theater, or a head-mounted display (HMD), for example a pair of video glasses. The electronic visual display is preferably a 4K- and/or 8K-capable electronic visual display and/or a 3D electronic visual display designed for stereoscopic presentation.

The system according to the present disclosure further comprises a control unit which is connected to the display means, in particular for one-directional or bidirectional data transfer. The control unit is configured to receive a time-resolved OCT signal of a selected field of view of a sample from an OCT system. For example, the sample is an operating site on a patient, especially an eye in the case of ophthalmological operations. However, the sample may also be any other operating site, for example brain tissue in neurosurgery, tissue located in the ENT region in the case of ENT surgery or the gingiva, tartar or dental nerves in the case of dental surgery. The sample may likewise be any other tissue or preparation (in vivo, in vitro or in situ). The time-resolved OCT signal has preferably been captured by virtue of a light signal being created and steered in part as a sample beam to the sample by means of the interferometer and subsequently being superimposed in the interferometer on a reference beam, likewise created from the light signal, in order to create an interference pattern.

The control unit of the system according to the present disclosure is further designed to ascertain, on the basis of the received time-resolved OCT signal, a time-resolved OCT image having at least one object and having a virtual surface, with a reflection of the at least one object in the OCT image being ascertained on the virtual surface. In other words, a B-scan with a reflective effect is created as a volume cross section, i.e. as a selected area in the OCT volume. The virtual surface thus integrates reflections from the object in the volume. The virtual surface, preferably also referred to as a reflective B-scan, is visualized within the OCT volume in order not to divert the attention of the surgeon from the focal region. The reflection is preferably calculated in real time during the volume ray marching and therefore, when applied to 4D-OCT, shows a reflected view of the object.

The control unit of the system according to the present disclosure is also configured to display the ascertained time-resolved OCT image on the display means. In other words, the control unit is configured to control the display means to display, on the display means, the time-resolved OCT image having the at least one object and having the virtual surface and also the reflection of the at least one object on the virtual surface. Here, the display is advantageously effected with the at least one object and with the virtual surface and also with the reflection of the at least one object on the virtual surface. The virtual surface facilitates the interpretation of the OCT image here; in particular, it enables a better understanding of distances between objects and areas within the OCT image. As a rule, surgeons operate with a stereoscopic microscope with which they view an operating site exclusively from an overhead perspective and manipulate anatomical structures with a precision in the submillimeter range. In the attempt to improve the surgical visualization, optical coherence tomography has been integrated into surgical microscopes which enable high-resolution, depth-resolved imaging. Advances in spiral scanning and swept source OCT even have leveled the way for volumetric imaging in real time and enable 4D visualizations of anatomical structures and surgical instruments. In such systems, GPU-accelerated direct volume rendering (DVR) has proven to be an effective method for visualizing surgical maneuvers, which enables a real-time representation of 4D-OCT data on stereo displays. Instead of viewing the operating site from above as in the case of stereoscopic microscopes, the depth-resolved properties of 4D-OCT can be used directly and completely if oblique or even more extreme lateral views are provided. This could even lead to more precise tool-tissue interactions since the visualization of the operating site from alternative viewing angles enables an improved distance perception. On the other hand, such arrangements also entail new complexities, since the surgical interaction from a lateral perspective is not customary in the current ophthalmological methods. In particular, hand-eye coordination is challenging when the instruments are navigated to a target from an unusual perspective, and this places a higher mental demand on the surgeon. An example of this is the lateral perspective, which can be advantageous for gripping a membrane during retinal peeling since it naturally reveals the distance between the surgical instrument and the retina or a retina membrane. Although this view permits a more precise instrument maneuver, the navigation of the instrument to a specific B-scan cross section is mentally more demanding than in the conventional view from top to bottom. The scope of this invention provides a form of virtual augmentation that is integrated into 4D-OCT visualizations in order to reduce the cognitive burden of the surgeon and improve the spatial understanding from desired perspectives such that the potential of 4D-OCT can be better utilized. In other words, displaying the time-resolved OCT image having the at least one object and having the virtual surface and also the reflection of the at least one object on the virtual surface on the display means constitutes an intelligent virtual B-scan mirror (IVBM), by virtue of a virtual mirror that represents a virtual B-scan being integrated into a selected volume cross section in the 4D-OCT. This provides a novel visualization concept for improving the targeted instrument interaction in 4D-OCT-guided surgery. Capturing an object positioned arbitrarily in space is a challenge for the surgeon when there are no perception cues. However, the reflection offers an additional view which considerably facilitates navigation to the target. The system according to the present disclosure therefore advantageously enables an optimal visualization of the OCT signal in accordance with the needs of the user, whereby the potentials of the OCT signal are optimally exploited, a maximum information content is made available to the user, and incorrect interpretations are avoided.

The control unit is preferably designed to control an OCT system to capture a time-resolved OCT signal from a selected field of view (region of interest-ROI) of a sample, with the field of view preferably being selected by a user. Further preferably, the system according to the present disclosure comprises an OCT system. The OCT system preferably comprises a light source, for example a broadband light source, designed to illuminate a sample. This light source preferably is a tunable (swept source) laser, for example a broadband laser, a supercontinuum laser, and/or an ultrashort pulse laser. In this case, a tunable laser at any given time can be a narrowband light source, the central frequency of which however can be varied over time in a targeted manner, or be formed from a plurality of narrowband light sources. However, other broadband sources can also be used, for example a superluminescent diode, for example in FD-OCT.

Further, the OCT system preferably comprises an interferometer, for example a Michelson, Mach-Zehner, or Koster interferometer, designed for creating and superimposing a sample beam and a reference beam. The interferometer preferably comprises a beam splitter for creating and superimposing the sample beam and reference beam from the light from the broadband source, a reference beam path, and a sample beam path. With further preference, the interferometer comprises means for setting an examined sample depth. Depending on the measurement method, this may be a means for creating a path difference (for instance, a mirror displaceable in the reference beam in the case of SD-OCT), a means for separating light of a specific path difference (for instance, an optical grating in the case of FD-OCT), or means for creating light with a specific path difference (for instance, a tunable source in the case of swept source OCT).

The OCT system further comprises a scanning mechanism designed to raster-scan the sample using the sample beam. In particular, the scanning mechanism is designed to raster-scan the sample beam over the sample in two dimensions. The scanning mechanism preferably is a scanning mirror, but other scanning mechanisms can also be used, for example an optical fiber scanner, a prism scanner, a Palmer scanner, or the like. It is possible to manage without a scanning mechanism in the case of an OCT system configured for full-field OCT.

The OCT system further comprises a detector designed to capture an interference pattern created by the superposition of sample beam and reference beam. The detector is for example a line detector, a two-dimensional detector array, a photodetector, or a dispersive detector. For example, the detector is designed as CCD or as a CMOS detector.

The OCT signal preferably is an interference signal, with the modulation of the envelope of the interference signal encoding reflection properties of the sample. The scanning mechanism allows the sample to be raster-scanned in two dimensions at a sample depth set by way of the path difference. A clock frequency (image refresh rate) for the time-resolved OCT signal arises from the utilized scanning mechanism, the utilized means for selecting or creating the path difference, for example an adjustable mirror in the reference beam, an optical grating in front of the detector or a tunable broadband light source, and the refresh rate of the detector.

The control unit computationally ascertains a time-resolved OCT image on the basis of the OCT signal, preferably by means of volume ray casting, volume rendering, ray tracing and/or ray marching, wherein, according to the invention, a time-resolved OCT image having the at least one object and having the virtual surface is ascertained, wherein a reflection of the at least one object in the OCT image is ascertained on the virtual surface.

In a preferred configuration of the system according to the present disclosure, the reflection of the at least one object on the virtual surface is ascertained when a ray emanating from an observation point and reflected by the virtual surface intersects the at least one object. For example, in the case of ray casting in direct volume rendering, the reflection of the at least one object on the virtual surface is ascertained by virtue of a ray reflected by the virtual surface being emitted in the direction of the at least one object, and if the ray emanating from the observation point and reflected by the virtual surface (i.e. a ray from an observation or viewing direction of the volume rendering) intersects with the at least one object. The reflection is then created on the virtual surface if there is a point of intersection between the ray reflected by the virtual surface and the at least one object.

By preference, the reflection of the at least one object on the virtual surface is only ascertained if the ray emanating from an observation point and reflected by the virtual surface intersects the at least one object within a predefined distance. In other words, the virtual surface is then sensitive only to an object and/or voxel assigned thereto, which are/is located in a predefined proximity, for example within a distance d, of the virtual surface and connected to the object.

In a likewise preferred embodiment of the system according to the present disclosure, the virtual surface is formed by a plurality of voxels, and the time-resolved OCT image on the virtual surface is ascertained on the basis of an OCT signal assigned to voxels that form the virtual surface. For example, during the volume rendering such as the volume ray marching, voxels having a position described by three coordinate axes are integrated into the virtual surface should these voxels form the virtual surface. Only constituents of the OCT signal that are assigned to these voxels are used for this purpose. It is thus possible to precisely form the virtual surface on the basis of selected signals and create the time-resolved OCT image in accordance with specific criteria. Moreover, it is particularly easily possible to check whether and which voxels are integrated in the virtual surface and contribute to ascertaining the time-resolved OCT image.

In a particularly preferred embodiment of the system according to the present disclosure, an OCT signal assigned to voxels that form the virtual surface is preferably highlighted in the OCT image. Accordingly, in other words, volume intensities integrated in the virtual surface are visually emphasized on the virtual surface in the OCT image. In other words, this is effected by virtue of voxels that are integrated into the virtual surface being emphasized, for example in volume rendering. Preferably, in addition, target structures that are aligned with the virtual surface are enhanced. These target structures preferably include, inter alia, anatomical target structures such as a macular hole, a point on the retina at which the peeling should be initiated, or a retina membrane after the peeling has been initiated. These are then particularly preferably highlighted even more in relation to the remaining voxels that form the virtual surface. The virtual surface can be described mathematically, and so a check as to whether voxels are assigned to the virtual surface and additionally to target structures is possible particularly easily, and the corresponding voxels are enhanced in the OCT image on the basis of the result. Preferably, highlighting is effected in the OCT image by virtue of a dynamically modifiable parameter being used to enhance the corresponding voxels and being taken into account in an overall appearance of the voxels that form the virtual surface. Moreover, perception cues are made possible by virtue of a point of intersection of the at least one object with the cross section being visualized and enhanced if the object or voxels that are assigned to the object are additionally assigned to the voxels that form the virtual surface.

Further preferably, the time-resolved OCT image on or at the virtual surface is furthermore ascertained on the basis of an OCT signal assigned to voxels that are arranged behind the virtual surface in an observation direction. As a result, volume structures behind the plane are retained in the OCT image. Voxels not included in but behind the virtual surface are rendered using the direct volume rendering convention known to a person skilled in the art. This is preferably implemented using an adaptive opacity transfer function, which is applied to voxels that are arranged behind the virtual surface in the observation direction. These corresponding voxels are preferably ascertained by checking whether a voxel is present behind the virtual surface along an observation direction. As a result, a plastic view is maintained, and the view of the user is not impaired. Essential components that would otherwise be concealed by the virtual surface are thus still displayed, and so all important information is retained.

In a likewise preferred embodiment of the system according to the present disclosure, an OCT signal assigned to voxels that are arranged behind and/or in front of the virtual surface in an observation direction is preferably deemphasized in the OCT image. In other words, the virtual surface is supplemented by a type of semi-transparency, whereby the background is maintained and not concealed by the virtual surface. The transparency value can assume values between 0 and 100%, whereby transparency is created in accordance with the specific selection for the OCT signal assigned to voxels that are arranged behind and/or in front of the virtual surface in the observation direction. Accordingly, the characteristic of the transparency is selected in order to create a desired visual appearance. In particular for surgical procedures under the retinal surface, such as e.g. subretinal and intraretinal injections, the volume assigned to voxels that are arranged in front of the virtual surface in the observation direction is depicted semi-transparently by attenuation, and so the reflection of the at least one object on the virtual surface is visible through the retina. Consequently, the option of displaying further helpful information is supplemented by a created transparency of the virtual surface on account of the attenuation, since, firstly, structures behind the surface remain visible and, secondly, structures in front of the virtual surface are displayed in a transparent manner.

The at least one object preferably comprises a medical instrument and/or an anatomical structure. The virtual surface accordingly acts as an intelligent mirror by virtue of being sensitive only to voxels which are connected to a medical instrument and/or an anatomical structure. The reflection of these objects makes it considerably easier for the surgeon, for example, to detect and estimate distances and directions of movement. The reflection of a medical instrument makes a further orientation aid available to them without having to give the plan view of the field of view. As a result, the advantageous view for effective hand-eye coordination is maintained, and at the same time, for example, improved perception of distances is made possible. The distance to a target, for example when approaching small structures located on or above the retina within the scope of peeling the epiretinal membrane (ERM) or the internal limiting membrane (ILM), can be represented with minimal magnifications and without additional images or viewing angles, which would divert the visual focus of the surgeon from the target region.

In a preferred embodiment of the system according to the present disclosure, the control unit is furthermore configured to ascertain the at least one object by segmenting the OCT signal or the OCT image. In this case, the segmentation is implemented in particular before the creation of the at least one object, on the basis of the OCT raw data, and/or on the basis of an OCT image ascertained therefrom. As a result of the segmentation of the OCT signal, the control unit is advantageously able to identify the position of the at least one object in the OCT volume. A person skilled in the art knows of various algorithms for automatically segmenting objects in image data, and these algorithms are presently used by the control unit in order to ascertain the reflection of the at least one object. These algorithms include both conventional threshold value-based image processing algorithms and more recent 2D and 3D segmentation networks that are based on machine learning (for example, a U-Net segmentation network). The control unit is preferably designed to obtain the segmentations from individual 2D OCT B-scans or directly from the entire 3D OCT volume. The positions obtained in the process are advantageously usable in volume rendering, ray tracing, and/or ray marching, as described hereinabove.

According to this preferred embodiment, one or more objects of interest are initially identified in part or in full with the aid of a segmentation method. The reflection is then implemented only for the objects of interest.

Preferably, the segmentation comprises a combination of a neural convolution network and conventional image processing. As a result of this, it is possible to obtain real-time performance.

The OCT signal particularly preferably comprises a multiplicity of A-scans, and the control unit is configured to ascertain an en-face view (plan view) on the basis of the multiplicity of A-scans, to ascertain the at least one object by segmentation in the en-face view, to ascertain, from the multiplicity of A-scans, a plurality of A-scans that contain the at least one object, and to ascertain a position of the at least one object in the depth direction in the plurality of A-scans. In other words, an integration of intensities along the A-scans is carried out when ascertaining the en-face view. Subsequently, the at least one object is then ascertained in the en-face view by segmentation, preferably by comparing the integrated intensities with a threshold value, with the object being ascertained provided the predefined threshold value is exceeded. Preferably, this takes place during the ray marching along a ray emanating from an observation point and reflected by the virtual surface and the checking for a point of intersection. Preferably, the threshold value is ascertained empirically in order to discard OCT speckle noise and in order then to determine whether the scanned point should be assigned to the object. The number of scanned steps along the reflected ray is limited in order to preferably reflect the object only if it is in the vicinity of the virtual surface. Thereafter, a plurality of A-scans are ascertained from the multiplicity of A-scans if they contain the at least one object. Furthermore, a position of the at least one object is ascertained in the depth direction in each A-scan of the plurality of A-scans. Since the virtual surface acts as a mirror for the at least one object, it is necessary, in other words, to identify mirror candidate voxels in the volume. In order to achieve the real-time processing rates required for 4D-OCT visualizations, the instrument is preferably initially identified in a 2D projection image of the volume. Thus, a 2D projection image is produced that encodes the average intensity along each OCT A-scan. This image is forwarded to a convolutional neural network, preferably U-net or a U-net-like convolutional neural network, in order to create a binary object map. Since the OCT signal at the object surface is completely blocked in the case of a medical instrument, for example, anatomical structures below the instrument are rendered unrecognizable, and the corresponding A-scans contain only object-related voxels. The 3D position of the voxels associated with the object can therefore be ascertained on the basis of the detected A-scans in the binary object map. Before the OCT volume and the object map are transmitted to a volume rendering algorithm, the volume is preferably additionally processed by applying a 3D median filter to reduce speckle noise in the OCT.

Preferably, the segmentation comprises the assignment of voxels to the object. In addition to information about the position or pose and also an intensity or a scatter value, voxels accordingly comprise information about an assignment to the object. This is preferably effected in binary form, with a binary value confirming or denying an assignment. The determination of the assignment is preferably implemented by comparing an intensity of a voxel with a predefined threshold value. If the latter is exceeded, for example, an assignment to the object is assumed.

The control unit according to the present disclosure is further preferably designed and configured to ascertain or segment the at least one object on the basis of object-specific information. In other words, additional information that characterizes the object can be used for the identification and segmentation of this object. For example, this information is information relating to the size, geometry, estimated relative pose and/or material of the object. The object-specific information further preferably also comprises information relating to a surgical phase (a phase of a specific operative procedure), in which the segmentation of the respective object is desired. In combination with an algorithm for identifying the phase, a variable segmentation of objects can consequently be implemented fully automatically.

The system according to the present disclosure preferably further comprises an interface designed to capture a user input. The interface preferably is a hand switch, a foot switch, and/or means for recognizing a head movement and/or eye movement or an actual viewing direction, for example integrated into video glasses or into a head-mounted display, HMD. The interface may further be designed to capture voice commands and may comprise at least one microphone to this end. Likewise preferably, the interface is a keyboard, a joystick, a mouse, a touchscreen, or a combination thereof.

According to this preferred embodiment, the control unit is further designed to control the interface for acquiring a user input. Further preferably, the user input is preceded by a prompt for input, output to the user by means of the output means for example. Likewise preferably, the user input is a selection of a plurality of predefined input options, which for example specify a virtual illumination direction and/or a procedure to be carried out. Likewise preferably, the user input is a user input which is freely definable by the user within a given scope. Likewise preferably, the control unit is designed to assist the user with the user input, for example by displaying a plurality of dialogs for the targeted query of specific user inputs.

According to this preferred embodiment, the control unit is further configured and designed to segment a multiplicity of objects in the OCT signal or the OCT image. In other words, there is no targeted segmentation (for example by means of object-specific information), but all objects in the OCT signal or OCT image are initially segmented. The control unit is further designed to ascertain or select the at least one object from the multiplicity of segmented objects on the basis of a user input received by way of the interface. Consequently, the reflection may be produced only for a single object of interest, for example for a surgical tool, or else for a plurality of objects of interest, for example for a plurality of surgical tools or anatomical structures.

In a likewise preferred embodiment of the system according to the present disclosure, the control unit is configured to ascertain a color coding of the reflection of the at least one object on the basis of a distance between the at least one object and the virtual surface in the time-resolved OCT image. Accordingly, in the integration of the reflection of the at least one object, a color space is used to encode the distance between the reflected object and the virtual surface. Preferably, the distance is ascertained on the basis of a ray that intersects the at least one object, is coded on the basis thereof by using a color coding, emanates from an observation point and is reflected by the virtual surface. The color coding preferably comprises the L*a*b* color space, preferably a perceptually linear color space. A color space is perceptually linear if a change in length in any direction X of the color space is perceived as the same change by a human. A perceptually nonlinear color space can have strong contrasts at the transition from one hue to another hue. In the data visualization, these contrasts can erroneously be regarded as changes in the data and not as transitions in the color palette. In other words, a perceptually linear color space causes the same geometric distance at each location of the color space to reflect the same amount of perceived color differences. An improved spatial representation of the at least one object is possible as a result. By way of example, the object is a medical tool, the spatial pose of which can be classified and estimated considerably better on the basis of the color coding.

Further preferably, the control unit is configured to vary a position and/or orientation of the observation point, the observation direction and/or the virtual surface. As already mentioned, the rendering of 3D OCT volume images may lead to problems with regards to the correct interpretation of spatial pose relationships between objects/areas in the OCT image. For example, the relative pose of a surgical tool tip and an anatomical structure may be interpreted incorrectly, depending on the viewing angle. This problem is solved by positioning a virtual surface. The virtual surface comprises the reflection of the at least one object, with both the virtual surface and the reflection thereon being dependent on the position and/or orientation of the observation point, the observation direction and/or the virtual surface itself. The positional perception of objects of interest in the OCT image and the estimate of the distance thereof from anatomical structures can be assisted by varying position and/or orientation of the observation point, the observation direction and/or the virtual surface. Likewise preferably, the control unit is configured to display the position and/or orientation of the observation point, the observation direction and/or the virtual surface in the time-resolved OCT image. Advantageously, this additionally assists the user's understanding of the created reflection on the virtual surface.

According to this embodiment, too, the system according to the present disclosure preferably comprises an interface designed to capture a user input. The interface preferably is the interface already described above. The control unit then is preferably configured to set the position and/or orientation of the observation point, the observation direction and/or the virtual surface on the basis of the user input. The position and/or orientation of the observation point, the observation direction and/or the virtual surface can thus advantageously be set manually in order to adjust these to the field of view or the focus region of the surgeon and to present a perspective which is particularly suitable for them. This allows the user to carry out precisely the manipulations that they consider helpful for their personal understanding.

As an alternative or in addition, the system according to the present disclosure further comprises a device interface designed to capture a device parameter. The device interface is preferably an interface for connecting a surgical-microscopic system, as still described in detail hereinbelow. Likewise preferably, the device interface however is also an interface for connecting any other imaging system. Further, the device interface can be an interface for connecting a medical instrument. Likewise preferably, a system for tracking a medical or medical engineering instrument is connectable via the device interface, wherein the tracking system may also be the surgical-microscopic system or any other imaging system. By way of example, the medical instrument is a pointer, a probe, a pair of tweezers, an awl, a phaco tip, an endoscope, and endo LED of a gripper or the like. According to this embodiment, the controller is preferably configured to communicate, in particular communicate bidirectionally, with the device interface and further configured to control the device interface to capture a device parameter of a device connected by means of the interface.

According to this embodiment, the control unit is further designed to ascertain the position and/or orientation of the observation point, the observation direction and/or the virtual surface on the basis of a device parameter captured by means of the device interface.

In a likewise preferred embodiment of the system according to the present disclosure, the at least one object further comprises a medical instrument. Preferably, the device parameter describes a pose, a position, and/or a state of a connected medical (or a tracked medical) instrument or of a medical (or a tracked medical) instrument tracked by means of the tracking system. By way of example, the medical instrument is a probe, a pointer, a pair of tweezers, an awl, a phaco tip, an endoscope, an endo LED or the like.

In this case, a type of the medical instrument can preferably be implemented on the basis of a device interface for connecting the medical instrument, by means of a tracking system (for example by identifying a target ID), and/or on the basis of an input via a user interface. Likewise preferably, a type of the medical instrument introduced into the field of view of the surgical-microscopic system is identified by an image analysis of the video image data, for example by means of segmentation and object recognition. A position of the medical instrument is preferably ascertained on the basis of the detection of a marker and/or a target comprising a plurality of markers, wherein the marker may be a label on or a structure of the medical instrument. The marker and/or the target are preferably detected by means of the surgical-microscopic system and optionally using additional light sources (for example infrared LEDs) and/or following a registration/calibration of the medical instrument (for example by positioning the one tip of the medical instrument at a defined location). The spatial pose of the medical instrument may also be ascertained by the instrument itself. Advantageously, the position and/or orientation of the observation point, the observation direction and/or the virtual surface in this embodiment can be set automatically on the basis of the distance of the instrument from the eye anatomy or other regions of interest of the OCT image. For example, with a decreasing distance of the medical instrument from the virtual surface and/or, for example, an anatomical tissue, the position and/or orientation of the observation point, the observation direction and/or the virtual surface can be arranged at a decreasing distance from these regions, such that an adjustment is carried out in accordance with the current situation or the current process, and a suitable highlighting, transparent presentation and advantageous reflection for better spatial perception is carried out for this situation.

A state of the medical instrument introduced into the field of view of the surgical-microscopic system is likewise preferably ascertained on the basis of an image analysis of the video image data. By way of example, whether tweezers are opened or closed can be identified on the basis of the image data. Moreover, a user input for changing a state can be read by the control unit; for example, a user input for activating a phaco tip signals a change in the state thereof. Further, a sensor attached to the medical instrument can detect the change in the state of the latter, for example tweezers being closed, and can transmit a corresponding sensor signal to the control unit. A device parameter relating to the state of a connected medical instrument, received via the device interface, preferably also comprises the actuation of an input means of the medical device, with the result that the device parameter in this case also represents a user input at the same time. Likewise preferably, different device parameters are considered together, for instance the pose of the medical instrument (for example ascertained by the instrument itself and/or by a tracking system) and the actuation of an input means of the medical instrument. Thus, for example, a sequence of position and/or orientation of the observation point, the observation direction and/or the virtual surface corresponding to a sequence of poses of the instrument is advantageously settable. A device parameter describing the medical engineering or medical instrument is received via the device interface from the instrument itself or from a tracking system.

According to this preferred embodiment, the control unit is further configured to ascertain the position and/or orientation of the observation point, the observation direction and/or the virtual surface on the basis of the position, the type, and/or the state of the medical instrument. Thus, the position and/or orientation of the observation point, the observation direction and/or the virtual surface can be ascertained, for example, in a manner corresponding to the pose of a pointer or in a manner corresponding to the state of a gripper. A change in the state of a medical instrument of a specific type and/or at a specific location is preferably indicative of a specific phase of an operation. Consequently, recognizing the position, the type, and/or the state of the medical instrument can be used to choose the optimal position and/or orientation of the observation point, the observation direction and/or the virtual surface for this phase. This optionally also comprises an adjustment of further display parameters of the OCT images, for example a zoom level, a virtual viewing direction, a displayed depth, a cut direction, etc.

By way of example, using the aforementioned information, the control unit is able, either automatically or on the basis of a user input, to ascertain that a performed procedure is membrane peeling by means of tweezers and, in this case, to further ascertain a distance or a position of the tweezers relative to the virtual surface and/or the eye and to adjust or set the position and/or orientation of the observation point, the observation direction and/or the virtual surface on the basis of the aforementioned ascertainments. In a further example, the control unit is preferably able, either automatically or on the basis of a user input, to ascertain that a performed procedure is a subretinal injection of a “bleb” or a placement of a retina stent and, on the basis thereof, adjust the position and/or orientation of the observation point, the observation direction and/or the virtual surface in such a way that a user is assisted with the positioning of the needle or the stent, for example by virtue of the virtual surface being positioned with the smallest possible distance from the retina or a point of intervention and hence the reflection being placed flush with the real intervention location, with the result that a user is able to make an optimal estimate as to when the needle is placed correctly.

Likewise preferably according to this preferred embodiment, the control unit is configured to ascertain a target structure on the basis of anatomical information, and to set the position and/or orientation of the observation point, the observation direction and/or the virtual surface on the basis of the ascertained target structure. Known algorithms for the recognition of target structures on the basis of anatomical information can be used for the recognition thereof. These target structures include, inter alia, anatomical target structures such as a macular hole, a point on the retina at which the peeling should be initiated, or a retina membrane after the peeling has been initiated. These are accordingly ascertained in order then, for example, to position and align the virtual surface in such a way that the time-resolved OCT image on the virtual surface is ascertained on the basis of an OCT signal assigned to voxels that form the virtual surface and comprises these target structures. In this case, the voxels that form the virtual surface simultaneously comprise the target structure and are preferably highlighted, and so this positioning of the virtual surface provides a particularly informative and helpful view.

In a preferred embodiment of the system according to the present disclosure, the virtual surface comprises a concave or convex surface. In the case of a concave virtual surface, the latter acts as a virtual concave mirror. The image produced depends decisively on the distance of the object from the concave mirror, and this should be taken into account accordingly. If the object is situated between mirror and focal point, a virtual, erect and magnified image is produced. Accordingly, the virtual surface on which the reflection of the at least one object is ascertained is preferably arranged in such a way that the object is arranged between the virtual surface and the focal point. In the case of a convex virtual surface, the latter acts as a virtual convex mirror. In this case, the focal point is situated behind the virtual surface. A virtual (apparent), erect, reduced image is produced. Accordingly, an image reduced in size is ascertained, with the viewing angle being increased. This should be preferred if much of the surroundings of the field of view is wanted to be seen.

Further preferably, the at least one object and/or the reflection of the at least one object in the time-resolved OCT image is ascertained in punctiform and/or schematic fashion. Accordingly, stitch points or regions and/or an outline of the object are ascertained and reproduced. As a result, it is possible to represent only relevant regions of the object and/or of the reflection. For example, in the case of a medical device as an object, only relevant parts of the object and/or of the reflection are imaged in that case, with other parts, which are without significance to the procedure or disturb the latter, for example, being displayed in transparent fashion. In the case of a schematic ascertainment and representation, by contrast, the entire object and/or the mirror thereof remains visible in the form of outlines, and thus shows an exact spatial orientation without being displayed plastically at the same time. As a result, the image can be reduced to essential elements, and so a greater focus on essential elements is not impaired or diverted.

In a particularly preferred embodiment of the system according to the present disclosure, said system further comprises an OCT system (as described above) and also a surgical-microscopic system designed to capture a time-resolved image signal from the selected field of view of the sample. The surgical-microscopic system comprises an image sensor, which is designed to capture a time-resolved image signal of the selected field of view (region of interest-ROI) of a sample, and preferably an optical unit, which for example may comprise an objective lens and an eyepiece but may moreover however comprise further components, in particular further lenses, mirrors, beam splitters and/or the like. The optical unit and the image sensor are preferably designed in integrated fashion, for example as parts of a camera of a surgical microscope. According to this embodiment, the control unit is further designed and configured to ascertain video image data corresponding to the captured time-resolved image signal. In particular, the time-resolved image signal is a multiplicity of signals assigned to surface elements of the sample, which are captured sequentially or simultaneously for a specific raster of the sample surface, wherein the raster is determined by a scanning mechanism and/or the image sensor. Further, the time-resolved image signal has a clock frequency (image refresh rate), which is determined by a scanning mechanism and/or the image sensor. From this image signal, the control unit creates video image data with a raster (resolution) and an image refresh rate suitable for display on the display means. The control unit is furthermore configured to represent the video image data on the display means, simultaneously or sequentially with the time-resolved OCT image and/or with the virtual surface.

A further aspect of the present disclosure relates to a method for visualizing OCT signals. The latter includes the method step of receiving a time-resolved OCT signal of a selected field of view of a sample from an OCT system (as described hereinabove). In this case, the OCT signal includes a multiplicity of tuples, which each contain or represent a volume element of the sample and a scattering intensity corresponding to the volume element. The method further comprises the step of ascertaining a time-resolved OCT image having at least one object and having a virtual surface, with a reflection of the at least one object in the OCT image being ascertained on the virtual surface. The method according to the present disclosure finally includes the display, on the display means, of the time-resolved OCT image having the at least one object and having the virtual surface. The method according to the present disclosure realizes the same advantages as the system according to the present disclosure, and, in this respect, reference is made to the explanations given above.

In a preferred implementation of the method according to the present disclosure, said method further includes the step of ascertaining the at least one object by segmenting the OCT signal or by segmenting the OCT image, preferably in each case using (on the basis of) object-specific information relating to the at least one object and/or a user input. Consequently, the reflection of semantically segmented objects is advantageously created on the virtual surface.

Further preferred implementations of the method according to the present disclosure correspond to further preferred embodiments of the system according to the present disclosure and realize the same advantages as the embodiments.

A further aspect of the present disclosure relates to a computer program comprising commands which, when executed by a control unit as described hereinabove, preferably of a surgical microscope as described hereinabove, cause the system or surgical microscope as described hereinabove to carry out the method according to the invention as described hereinabove. The computer program preferably comprises commands which, when executed by a control unit as described hereinabove, preferably of a surgical microscope, cause the system or surgical microscope as described hereinabove to carry out the method according to the invention, in accordance with one of the preferred implementations, as described hereinabove. In this case, the computer program according to the invention is preferably stored in a transitory memory, for example a RAM element, or in a non-transitory storage medium, for example a CD-ROM, a flash memory or the like.

Further preferred embodiments of the invention will become clear from the other features set out in the dependent claims and from the figures explained below. The various embodiments of the invention that are set forth in this application can advantageously be combined with one another, unless specifically stated otherwise.

### DESCRIPTION OF THE FIGURES

The invention will be explained below in exemplary embodiments with reference to the associated drawings, in which:

FIG. 1 shows a schematic illustration of a system for visualizing OCT signals according to a first embodiment;

FIG. 2 shows a schematic illustration of a system for visualizing OCT signals according to a second embodiment;

FIG. 3 shows a schematic illustration of the visualization of an OCT image having at least one object and having a virtual surface;

FIGS. 4a-4d show an illustration of the methodology of the visualization of an OCT image having at least one object and having a virtual surface; and

FIGS. 5-7 shows various views of a visualization of an OCT image having at least one object and having a virtual surface;

FIG. 8 shows a schematic flowchart of a method according to an implementation;

FIG. 9 shows a development of a distance error over time; and

FIG. 10 shows results of a NASA-TLX poll regarding various stress factors.

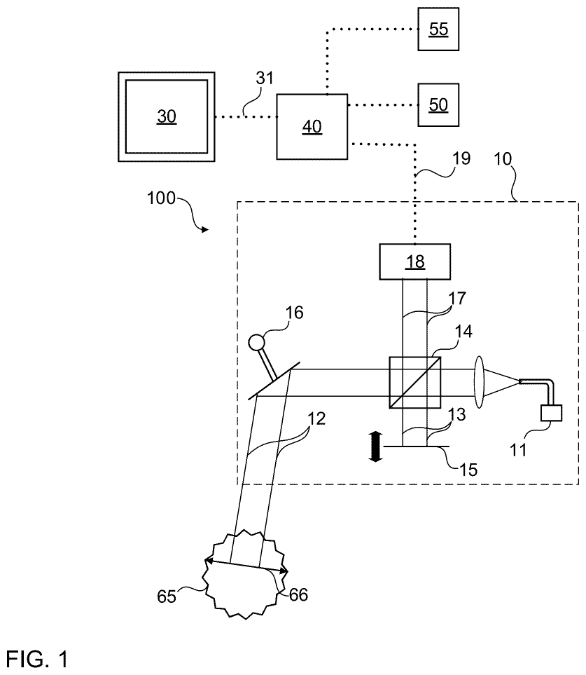

FIG. 1 shows a schematic illustration of a system 100 for capturing and visualizing OCT signals 19 according to a first embodiment.

The system 100 comprises an OCT system 10 having a broadband light source 11, for example a superluminescent diode. The light from the light source 11 is guided into an interferometer comprising a beam splitter 14 and a movable mirror 15. The light is split into a sample beam 12 and a reference beam 13 in the beam splitter 14. The sample beam 12 is raster-scanned over the sample 65 by means of a scanning mirror 16, with at least one portion of a chosen field of view 66 of the sample 65 being raster-scanned. The reference beam 13 is steered to the movable mirror 15 and reflected thereby back to the beam splitter 14. The sample beam 12 interacts with the sample 65, in particular with the volume of the sample 65, and is scattered back thereby to the scanning mirror 16, which steers the beam to the beam splitter 14. The back-scattered sample beam 12 and the reflected reference beam 13 are superimposed there, with a path difference between the superimposed beams 12, 13 being set by the movable mirror 15. The interference pattern 17 generated thus is captured by means of a detector 18, for example a CCD detector or a CMOS detector.

The time-resolved OCT signal 19 captured thus is transmitted from the detector 18 to the control unit 40. The control unit 40 is further connected to a user interface 50 for capturing a user input and to a device interface 55 for receiving a device parameter. For example, the device interface 55 serves to receive a device parameter of a device connected to the device interface 55, for example of a surgical-microscopic system 20, of any other imaging system, or of a medical instrument 70. Further, the device parameter may originate from a tracking system (not depicted here) connected to the device interface 55 but characterize the medical instrument 70, for example its spatial pose.

The control unit 40 ascertains a time-resolved OCT image 31 having at least one object 70 and having a virtual surface 60 for the captured time-resolved OCT signal 19, with a reflection 70′ of the at least one object 70 in the OCT image being ascertained on the virtual surface 60, and transmits the time-resolved OCT image 31 having the at least one object 70 and having the virtual surface 60 to the display means 30 for presentation.

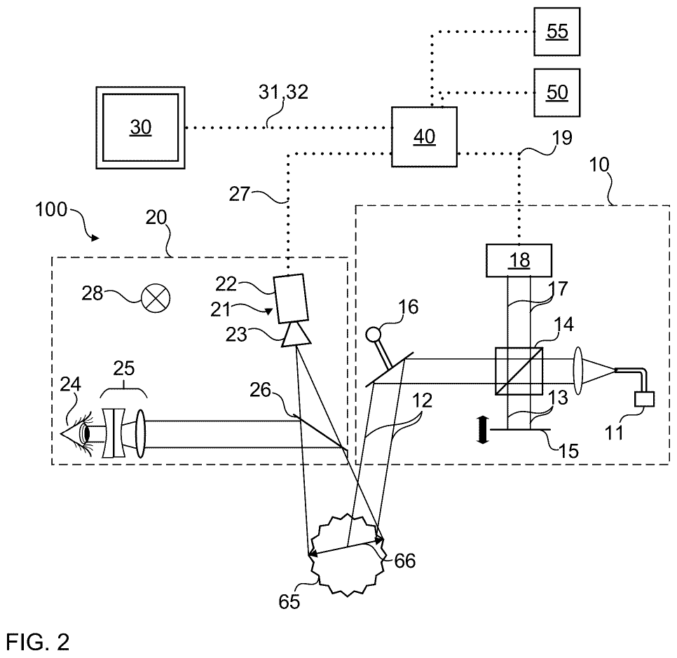

FIG. 2 shows a schematic illustration of a system 100 for capturing and visualizing OCT signals 19 according to a second embodiment. The same components are denoted by the same reference signs as in FIG. 1 and a repeated description of these components is therefore omitted.

The system 100 in FIG. 2 differs from that in FIG. 1 in that it further comprises a surgical-microscopic system 20 with a camera 21. The camera 21 comprises an image sensor 22 and an optical unit 23 and is designed to capture the field of view 66 of the sample 65 such that a time-resolved image signal 27 is captured as a result. The camera 21 can be a mono camera or a stereo camera. The control unit 40 ascertains video image data 32 that correspond to the captured time-resolved image signal 27. In the system 100 of FIG. 2, a time-resolved OCT signal 19 is captured in the same manner, and an OCT image 31 having the at least one object 70 and having the virtual surface 60 is ascertained therefrom, as has already been described with reference to FIG. 1. The video image data 32 are displayed simultaneously or sequentially on the display means 30 together with the OCT images 31, wherein a change between the OCT images 31 and the video image data 32 is implemented on the basis of a user input captured by means of the user interface 50, for example. The surgical-microscopic system 20 further comprises a piece of equipment for splitting the observation beam path 26, which splits the light beam transmitted from the field of view 66 of the sample 65 to the camera 23 and provides a component beam to the eye 24 of an observer via an eyepiece 25.

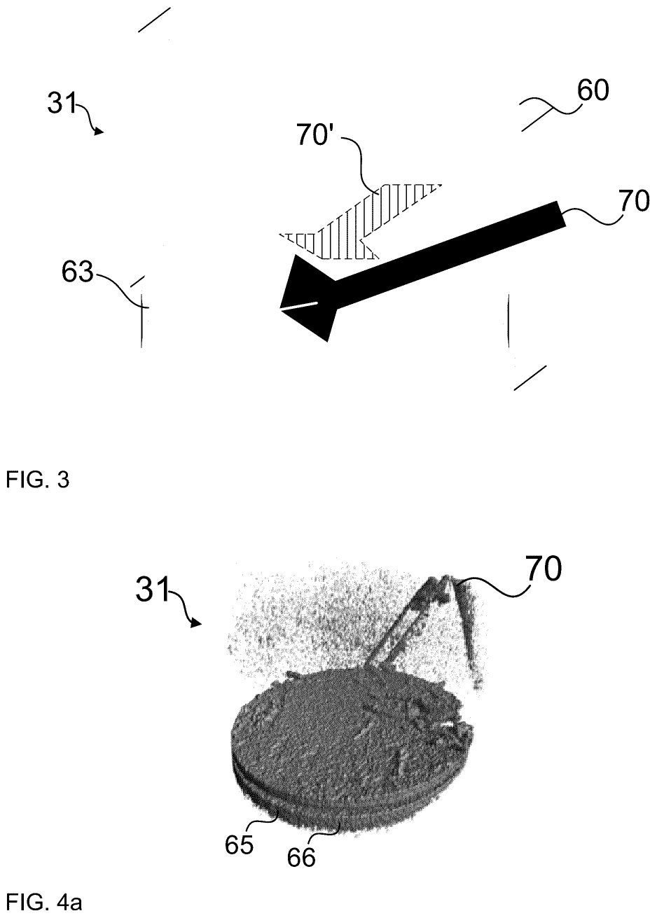

FIG. 3 shows a schematic illustration of the visualization of an OCT image 31 having at least one object 70 and a virtual surface 60. As described, the OCT image 31 is ascertained from the OCT signal 19 captured from the field of view 66 of the sample 65, wherein an object 70, presently a medical instrument 70, is in the vicinity of the sample 65 while the signal is captured. The OCT image 31 comprises an image representation of an area 63 of the field of view 66 of the sample 65, an image representation of the medical instrument 70, and an image representation of an anatomical structure in the field of view 66 of the sample 65. The scanning mirror 16 is used to steer the light from the broadband light source 11 of the OCT system 10 to the sample 65 in such a way that the light beams are incident virtually parallel to one another and at right angles to the sample surface in the field of view 66.

The OCT image 31 further comprises a reflection 70′ of the at least one object 70 on the virtual surface 60. This was calculated as reflection of the medical instrument 70 on the virtual surface 60. Further, the virtual surface 60 is semi-transparent, and so the anatomical structure behind the virtual surface 60 still remains visible.

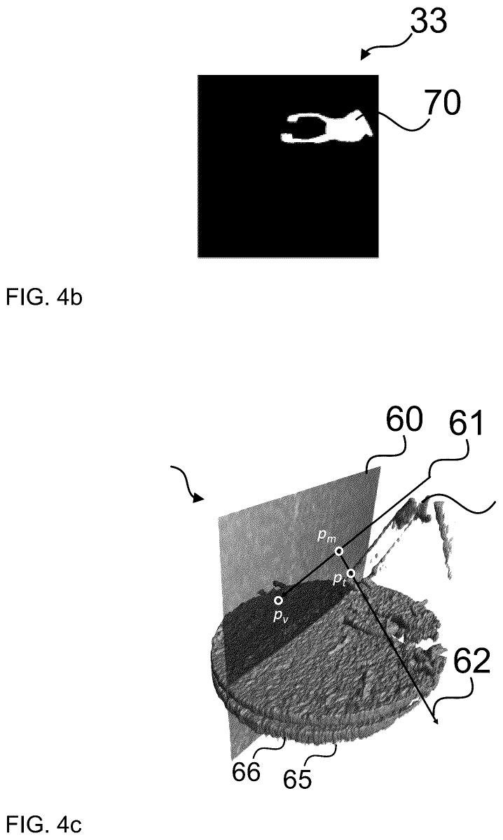

FIGS. 4a to 4d show a method according to the invention carried out by the system for visualizing OCT signals. FIG. 4a shows the original OCT volume. A virtual surface 60, alternatively also referred to as an intelligent virtual B-scan mirror (IVBM) 60, is ascertained as an extension of a selected virtual B-scan. According to the present preferred embodiment, the virtual surface 60 meets the requirements that voxels integrated in the IVBM 60 are highlighted in volume rendering while simultaneously being presented semi-transparently in order to obtain volume structures behind the IVBM 60, and that the selected B-scan cross section acts as an intelligent mirror by being sensitive only to voxels connected to the medical instrument 70 in the vicinity of the virtual surface 60. In the following paragraphs, the required components and also the structure and the direct integration of the IVBM 60 into a volume ray marching algorithm are described.

Since the virtual surface 60 integrates a mirror that is sensitive only to medical instruments, it is necessary to identify mirror candidate voxels in the OCT volume. In order to achieve the real-time processing rates required for 4D-OCT visualizations, the instrument 70 is initially identified in a 2D projection image 33 of the OCT volume. This is shown in FIG. 4b. A 2D projection image 33 or an en-face view 33 is ascertained, which encodes the average intensity along a multiplicity of OCT A-scans of the OCT signal. On the basis of this 2D projection image 33, the at least one object 70 is ascertained by segmentation in the en-face view. For this purpose, the 2D projection image is transmitted to a U-net-like convolutional neural network to create a binary instrument map Mₜₒₒₗ. Since the OCT signal 19 is completely blocked at the surface of the medical instrument 70, anatomical structures below the instrument 70 are rendered unrecognizable, and, from the multiplicity of A-scans, a corresponding plurality of A-scans that contain the at least one object 70 contain only instrument-related voxels. A position of the at least one object 70 in the depth direction in the plurality of A-scans in the form of a 3D position of the voxels associated with the instrument can therefore be ascertained on the basis of the detected plurality of A-scans in the binary instrument map Mₜₒₒₗ, as described below. Before the OCT volume and the binary instrument map Mₜₒₒₗ are transmitted to a suitable DVR algorithm, the OCT volume is additionally processed by applying a 3D median filter to reduce speckle noise in the OCT. With these components, the composition of the IVBM is realized completely within the DVR algorithm, as described in the following paragraph.

For this purpose, the relationships are illustrated in FIG. 4c. Three components contribute to the resulting beam color when an observation ray r_{B} 61 that corresponds to an observation direction starting from an observation point intersects with the virtual surface 60, as illustrated in FIG. 4c. Firstly, this is each voxel P_{M} along the observation ray r_{B} 61 that is assigned to and forms the virtual surface 60. Furthermore, this is each voxel Pᵥ that is arranged along an observation ray r_{B} 61 behind the virtual surface 60 in the observation direction. Then again, this is each specific voxel Pₜ assigned to the at least one object 70, at which a ray r_{M} 62 emanating from the observation point and extending along the observation ray r_{B} 61 and reflected by the virtual surface 60 intersects. In the present case, this is represented by the mirror beam r_{M} 62, which is reflected by the virtual surface 60 and intersects with the medical instrument 70.

According to this preferred embodiment, the present virtual surface 60 is formed as any desired plane which is selected either manually or automatically and which can thus be defined by the general equation:

$${xn}_{x} + {yn}_{y} + {zn}_{z} + d = 0 \qquad ( 1 )$$

where n=(nₓ, n_{y}, n_{z}) denotes the normal of the plane. Further, a distance threshold d_{ivBM} specifying the thickness of the virtual surface 60 is defined. During volume ray marching, a voxel P_{M} at the position (Pₓ,P_{y},P_{z}) is integrated into the virtual surface 60 should the following condition be satisfied:

$$\vert \frac{\vert {xn}_{x} + {yn}_{y} + {zn}_{z} + d \vert}{\sqrt{{n}_{x}^{2} + {n}_{y}^{2} + {n}_{z}^{2}}} \vert < {d}_{IVBM} \qquad ( 2 )$$

In order to visualize the reflections of the medical instrument 70, a mirror ray r_{M} 62 reflected by the virtual surface 60 is cast from each point P_{M}, which results from equation (2). The direction of the mirror ray is determined by:

$${r}_{m} = {r}_{B} - 2 * dot ( {r}_{B} , n ) * n \qquad ( 3 )$$

During volume ray marching along r_{M} 62, the binary instrument map Mₜₒₒₗ described at the outset and an intensity threshold value tₜₒₒₗ=0.25 (ascertained empirically in order to discard OCT speckle noise) are used to determine whether the sampled point is a mirror candidate:

$${M}_{tool} ( {P}_{t} ) = 1 ⋂ I ( {P}_{t} ) > {t}_{tool} \qquad ( 4 )$$

where I denotes an intensity of a voxel. The number of scanned steps n along r_{M} 62 is limited in order further to preferably reflect the medical instrument 70 only if it is in a defined vicinity of the virtual surface 60. In the case of an intersection with an instrument 70, as determined by equation (4), the volume ray marching is ended, and the reflection 70′ of the medical instrument 70 on the virtual surface 60 at P_{M} is supplemented with a color component Cpt. Preferably, the distance along r_{M} 62 between P_{M} on the virtual surface 60 and Pₜ on the medical instrument 70 is encoded by using a perceptually linear color map. Preferably, an L*a*b* color space having the following features is used for this purpose:

$${C}_{L * a * b *} ( I , {δ}^{*} ) = γ ( I ) * ( {δ}^{*} * {C}_{0} + ( 1 - {δ}^{*} ) * {C}_{1} ) \qquad ( 5 )$$

where δ*=(i_{M}*0.7)/nₛₜₑₚₛ is a distance attribute that takes into account the step index i_{M} ∈[0, nₛₜₑₚₛ] along r_{M} 62 when reaching the medical instrument 70 at Pₜ. Y (I) is a factor for scaling, which is also presented in “Layer-aware ioct volume rendering for retinal surgery.”, Weiss, J., Eck, U., Nasseri, M. A., Maier, M., Eslami, A., Navab, N., in: “Eurographics Workshop on Visual Computing for Biology and Medicine. The Eurographics Association”, Kozlfkova, B., Linsen, L., Vazquez, P. P., Lawonn, K., Raidou, R. G. (eds.) (2019). By selecting C₀=(I (Pₜ), −1.5,1) and C₁=(I (Pₜ), −1.0, −1.0), a color interpolation between a blue hue, when the medical instrument 70 is further away from the IVBM 60, and a green hue, when it is in the vicinity of the virtual surface 60, is achieved. The RGB component of the reflection 70′ of the instrument 70 is then obtained by:

$${C}_{Pt} = [ RGB ( {C}_{L * a * b *} ( I ( {P}_{t} ) , {δ}^{*} ( p ) ) ) ] \qquad ( 6 )$$

where RGB (C) is a conversion from L*a*b* into the RGB color space. In addition, the blurring of the reflection 70′ is reduced by

$$σ ( {P}_{t} ) = 1. - {( \frac{{step}_{m}}{{n}_{steps}} )}^{2}$$

as the distance of the medical instrument 70 from the virtual surface 60 increases.

The overall appearance of a voxel at P_{M}, which integrates the reflection 70′ and at the same time enhances the natural intensities on the virtual surface 60, is finally defined by:

$$C ( {P}_{m} ) = [ μ * I ( {P}_{m} ) * ( 1 - σ ( {P}_{t} ) ) + C ( {P}_{t} ) * σ ( {P}_{t} ) , α ( μ * I ( {P}_{m} ) ) ] \qquad ( 7 )$$

In practice, u is a dynamically modifiable parameter for enhancing the volume intensities of the virtual B-scan 60 and a (l) is a conventional piecewise linear blurring function. During volume ray marching, alpha blending is used to integrate the virtual surface 60 with the remaining volume structures according to equation (2). In general, voxels Pᵥ that are not included in the IVBM 60 can be rendered using any convention for direct volume rendering.

In our experiments, and for the visualizations in FIGS. 4 to 7, use is made of conventional Phong shading, as was used previously for the OCT volume representation. In the visualizations, it provides structural surface details and at the same time visually highlights the virtual surface 60 in the final rendering.

The result of the methodology is shown in FIG. 4d. It is clearly evident that the medical instrument 70 is ascertained and illustrated as a reflection 70′ on the virtual surface 60. Nevertheless, volume structures behind the virtual surface 60 are still recognizable.

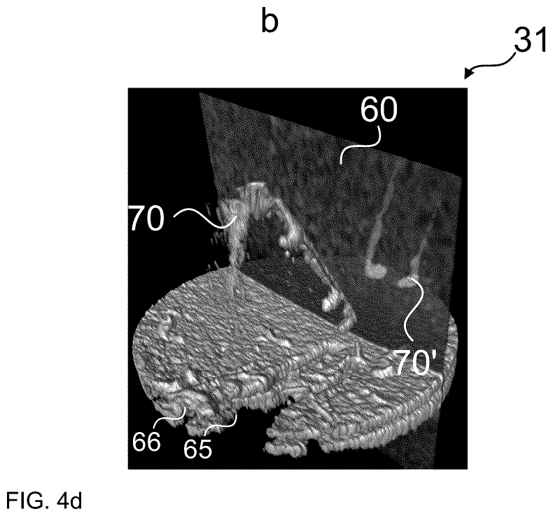

FIG. 5 shows another view of the OCT image 31. In that case, visual highlighting of volume intensities integrated in the virtual surface 60 is recognizable on the virtual surface 60 in the OCT image 31. In other words, voxels that are integrated into the virtual surface 60 are highlighted, for example in volume rendering. Further, target structures that are aligned with the virtual surface 60 are enhanced. These target structures preferably include, inter alia, anatomical target structures which are depicted in more highly illuminated form.

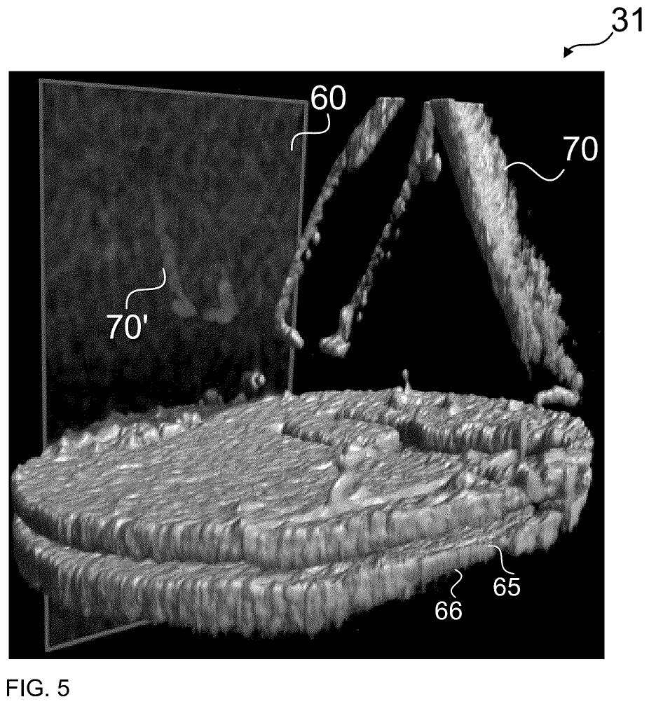

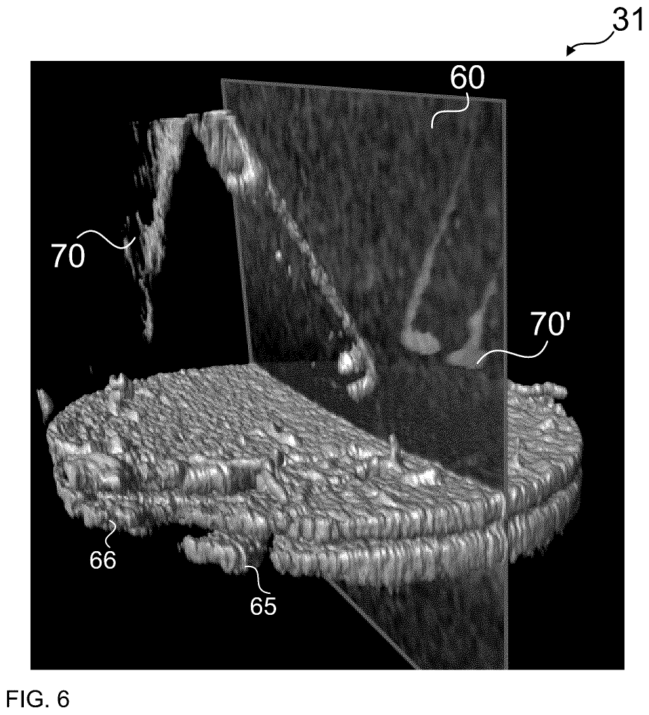

As illustrated in FIG. 6 and FIG. 7, the IVBM 60 has the advantage that it provides perception cues by visualizing the point of intersection of the instrument 70 with the virtual surface 60 as soon as the tip of the instrument 70 is incident on the tip of the reflected instrument 70′. It is also able to visualize instrument structures that are concealed in the direct view or are situated behind the IVBM 60 and enhances target structures that are aligned with the IVBM 60.

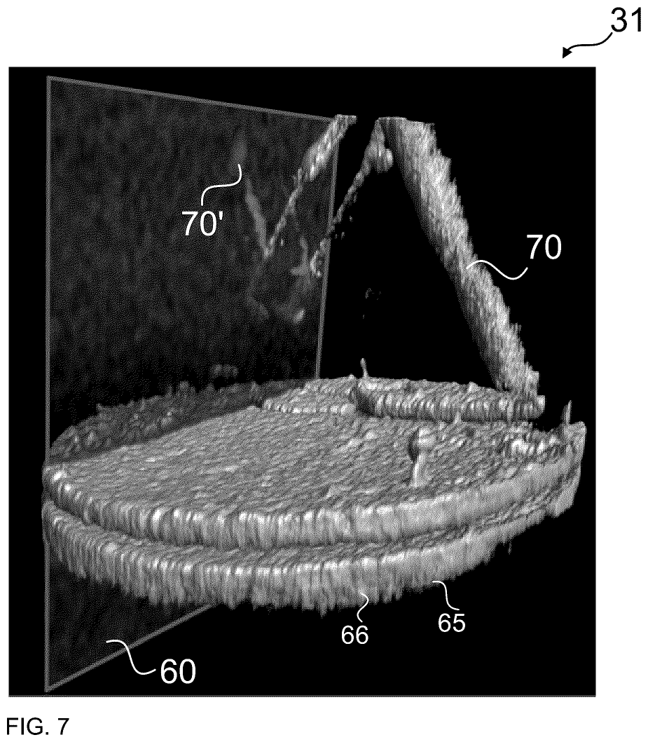

FIG. 8 shows a schematic flowchart of a method according to an implementation. The method includes a first step S100 of receiving a time-resolved OCT signal 19 of a selected field of view 66 of a sample 65 from an OCT system 10. In this case, the OCT signal 19 includes a multiplicity of tuples, which each represent a volume element of the sample 65 and a scattering intensity corresponding to the volume element. The method further comprises the second step S200 of ascertaining S200 a time-resolved OCT image 31 having at least one object 70 and having a virtual surface 60, with a reflection 70′ of the at least one object 70 in the OCT image being ascertained on the virtual surface 60. Finally, the time-resolved OCT image 31 is displayed on the display means 30 in a third step S300. Further, the inventors have evaluated whether IVBMs can be used for the live representation of 4D-OCT data by implementing the proposed concept in a visualization framework. The 2D projection image creation and IVBM-integrated volume ray marching were implemented with OpenGL 4.6 and tested on an NVidia RTX 3090Ti GPU. An instrument segmentation network was trained on a user-defined data record consisting of 3356 2D projection images created from OCT volumes with a resolution of 391×391×644 voxels, which contain a synthetic eye model and surgical tweezers and contain random rotations and reflections for data expansion. PyTorch 1.13 and TensorRT 8.4 were used for model training and optimization. The data were labeled by two biomedical technicians.

The average total processing and rendering time based on 20 test volumes with the same resolution was 44.3 (+3.1) ms (filter: 8.1 (+1.2) ms, creation of projection images and segmentation of the instruments: 5.7 (+2.6) ms, rendering: 30.5 (+0.6) ms). These benchmarks were achieved with nₛₜₑₚₛ=120 mirror sample steps. To demonstrate the 4D interactions, the method was integrated into a 4D-SS-OCT system made available by a medical technology company. OCT volumes were recorded with a resolution of 391×391×644 at volume refresh rates of 10 Hz.

In order to find out whether an IVBM can assist the users in performing targeted instrument maneuvers under 4D-OCT guidance, a user study was performed, in which the participants were asked to move the tip of a surgical instrument to defined target locations. In order to achieve continuous, accurate and efficient data capture during the study, use was made of a virtual environment with simulated 4D-OCT that employed the method proposed in Sommersperger, M., Martin-Gomez, A., Mach, K., Gehlbach, P. L., Nasseri, M. A., lordachita, I., Navab, N.: Surgical scene generation and adversarial networks for physics-based ioct synthesis. Biomedical Optics Express 13 (4), 2414-2430 (2022). In addition, a 3D haptic input device (3D Systems Geomagic Touch) was integrated to navigate the virtual surgical instruments, with use being made of 4:1 motion scaling in order to reduce the influence of the individual manual tremor of the users. Small targets were generated on the retina, and the IVBM was automatically positioned at the target locations. In order to measure the effectiveness of the IVBM in the interaction from unusual perspectives, the virtual scene was rendered from a fixed view that was approximately orthogonal to the A-scan direction. The renderings were displayed in a VR headset that used stereo rendering.

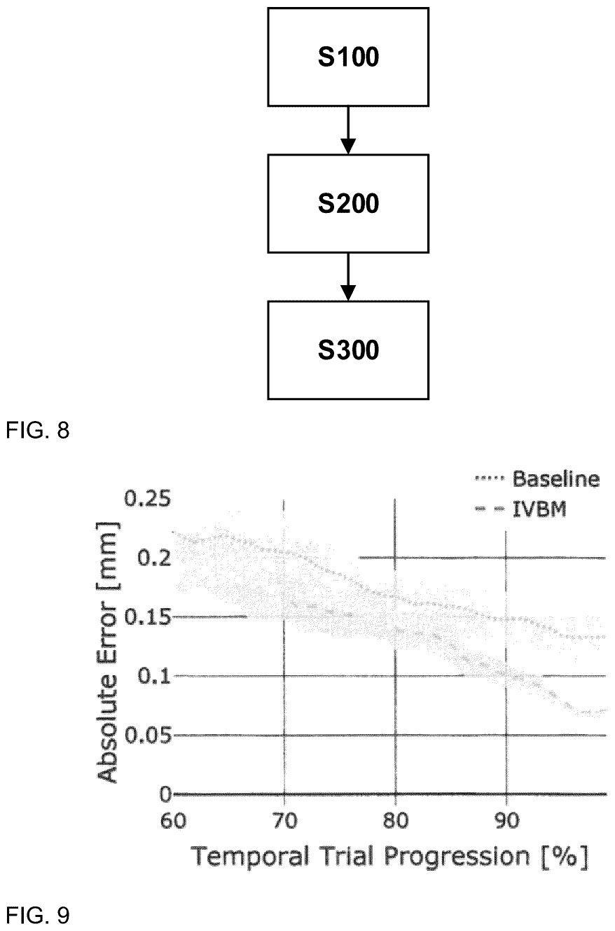

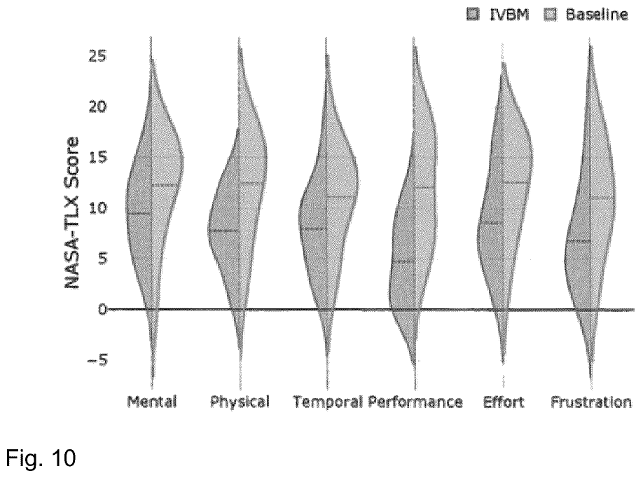

The users were prompted to navigate the instrument tip to the target location and push a button once they were satisfied with the positioning. The participants were a total of 15 biomedical experts (12 male, 3 female) familiar with ophthalmology and OCT. The study was performed in accordance with the Declaration of Helsinki, the study data were anonymized, and, before the study, vision tests were performed to ensure healthy vision of the participants. After the participants had familiarized themselves with the interaction in the virtual environment, they performed 8 trials, during which the IVBM was activated. For the ablation, the same number of trials were carried out without IVBM, with the method for 4D-OCT-DVR with Phong shading proposed in Viehland, C., Keller, B., Carrasco-Zevallos, O. M., Nankivil, D., Shen, L., Mangalesh, S., Kuo, A. N., Toth, C. A., Izatt, J. A., et al.: Enhanced volumetric visualization for real time 4d intraoperative ophthalmic swept-source oct. Biomedical optics express 7 (5), 1815-1829 (2016) serving as the basis. The accuracy of the positioning and the distance from the target were measured over the course of the trials. The results show that the users reached the target with an average error of 70 μm (+40 μm) when the IVBM was activated, while an average error of 135 μm (+128 μm) was measured in the base data rendering, indicating statistically significant differences between the distributions (p<0.002 based on a Kruskal-Wallis test upon detection of unequal variances). Moreover, the distance between the instrument tip and the target was analyzed with respect to the course of the trial. FIG. 9 shows that, in the case of activated IVBM, the deviation of the distance error decreased continuously, in particular in the last quarter of the course of the trial, which led to a more accurate and more precise target acquisition. The results of the NASA-TLX poll in FIG. 10, conducted after the study, showed improvements in all categories when the IVBM was activated. However, statistical significance could only be determined in the categories of performance (p<0.001) and physical demand (p<0.02) on the basis of an ANOVA test for equal variances.

If an IVBM was made available to the users, statistically significant improvements were found with regard to the target error. The results of the subjective evaluation of the task burden were also better overall if the IVBM was activated, but no statistical significance could be achieved in categories such as mental demand or frustration. Interestingly, a higher target accuracy was nevertheless achieved with the IVBM, even if the users indicated a higher effort or assessed their performance as worse in comparison with the initial value. An abstraction of the reflected view in order to integrate only important key points of the instrument could further reduce the mental demand on the users. An advantage of the IVBM is the direct in-situ visualization, which also highlights anatomical target structures, since the user need not turn their gaze away from the operating site in order to perceive the reflected view.

### LIST OF REFERENCE SIGNS

10 OCT system
11 Broadband light source
12 Sample beam
13 Reference beam
14 Beam splitter (interferometer)
15 Movable mirror (interferometer)
16 Scanning mechanism (scanning mirror)
17 Interference pattern
18 Detector
19 Time-resolved OCT signal
20 Surgical-microscopic system
21 Camera
22 Image sensor
23 Optical unit
24 Eye of a viewer
25 Eyepiece
26 Piece of equipment for splitting the observation beam path
27 Time-resolved image signal
30 Display means
31 OCT image
32 Video image data
33 2D projection image/en-face view
40 Control unit
50 User interface
55 Device interface
60 Virtual surface/IVBM
61 Observation ray r_{B}
62 Reflected ray/mirror ray r_{M}
63 Surface
65 Sample
66 Field of view
70 Object/medical instrument
70′ Reflection of the object/of the medical instrument

1. A system for visualizing OCT signals, comprising:
a display means designed for the time-resolved display of image data; and
a control unit, which is configured
to receive a time-resolved OCT signal of a selected field of view of a sample from an OCT system;
to ascertain, on the basis of the OCT signal, a time-resolved OCT image having at least one object and having a virtual surface, with a reflection of the at least one object in the OCT image being ascertained on the virtual surface; and
to display the time-resolved OCT image on the display means.

2. The system as claimed in claim 1, wherein the reflection of the at least one object on the virtual surface is ascertained when a ray emanating from an observation point and reflected by the virtual surface intersects the at least one object.

3. The system as claimed in claim 1, wherein the virtual surface is formed by a plurality of voxels, and the time-resolved OCT image on the virtual surface is ascertained on the basis of an OCT signal assigned to voxels that form the virtual surface.

4. The system as claimed in claim 1, wherein an OCT signal assigned to voxels that form the virtual surface is preferably highlighted in the time-resolved OCT image.

5. The system as claimed in claim 1, wherein the time-resolved OCT image on or at the virtual surface is furthermore ascertained on the basis of an OCT signal assigned to voxels that are arranged behind the virtual surface in an observation direction.

6. The system as claimed in claim 1, wherein an OCT signal assigned to voxels that are arranged behind and/or in front of the virtual surface in an observation direction is preferably deemphasized in the OCT image.

7. The system as claimed in claim 1, wherein the at least one object comprises a medical instrument and/or an anatomical structure.

8. The system as claimed in claim 1, wherein the control unit is configured to ascertain the at least one object by segmenting the OCT signal or the OCT image.

9. The system as claimed in claim 1, wherein the OCT signal comprises a multiplicity of A-scans, and the control unit is configured
to ascertain an en-face view on the basis of the multiplicity of A-scans,
to ascertain the at least one object by segmentation in the en-face view,
to ascertain, from the multiplicity of A-scans, a plurality of A-scans that contain the at least one object, and
to ascertain a position of the at least one object in the depth direction in the plurality of A-scans.

10. The system as claimed in claim 1, further comprising an interface designed to capture a user input, wherein the control unit is configured to segment a multiplicity of objects in the OCT signal or the OCT image and select the at least one object therefrom on the basis of the user input.

11. The system as claimed in claim 1, wherein the control unit is configured to ascertain a color coding of the reflection of the at least one object on the basis of a distance between the at least one object and the virtual surface in the time-resolved OCT image.

12. The system as claimed in claim 1, wherein the control unit is configured to vary a position and/or orientation of the observation point, the observation direction and/or the virtual surface.

13. The system as claimed in claim 12, wherein the system further comprises an interface designed to capture a user input, and the control unit is configured to set the position and/or orientation of the observation point, the observation direction and/or the virtual surface on the basis of the user input, and/or wherein the system further comprises a device interface designed to capture a device parameter, and the control unit is configured to set the position and/or orientation of the observation point, the observation direction and/or the virtual surface on the basis of the device parameter.

14. The system as claimed in claim 12, wherein the control unit is configured
to ascertain a target structure on the basis of anatomical information, and
to set the position and/or orientation of the observation point, the observation direction and/or the virtual surface on the basis of the ascertained target structure.

15. The system as claimed in claim 1, wherein the virtual surface comprises a concave or convex surface.

16. The system as claimed in claim 1, wherein the at least one object and/or the reflection of the at least one object in the time-resolved OCT image is ascertained in punctiform and/or schematic fashion.

17. The system as claimed in claim 1, further comprising:
an OCT system,
a surgical-microscopic system designed to capture a time-resolved image signal from the selected field of view of the sample,
wherein the control unit is further configured to ascertain corresponding video image data on the basis of the captured time-resolved image signal and to simultaneously or sequentially display the video image data with the time-resolved OCT image and/or with the virtual surface on the display means.

18. A method for visualizing OCT signals, including the method steps of:
receiving a time-resolved OCT signal of a selected field of view of a sample from an OCT system, with the OCT signal including a multiplicity of tuples each representing a volume element of the sample and a scattering intensity corresponding to the volume element;
ascertaining a time-resolved OCT image having at least one object and having a virtual surface, with a reflection of the at least one object in the OCT image being ascertained on the virtual surface; and
displaying the time-resolved OCT image on the display means.
